(19) United States
(12) Patent Application Publication
(10) Pub. No.: US 2012/0129819 A1
(43) Pub. Date: May 24, 2012

(54) GEL COMPOSITIONS FOR ADMINISTRATION OF PHARMACEUTICALLY ACTIVE COMPOUNDS

(75) Inventors: Thierry Vancaillie, Castlecrag (AU); Alan Hewitt, Wrexham (GB)

(73) Assignee: Medortus (UK) Ltd, Wrexham (GB)

(21) Appl. No.: 13/264,211

(22) PCT Filed: Apr. 14, 2010
(86) PCT No.: PCT/AU10/00408

§ 371 Date: Feb. 9, 2012

(30) Foreign Application Priority Data
Apr. 14, 2009 (AU) 2009901585

(51) Int. Cl. A61K 31/565; A61P 31/00; A61P 29/00; A61P 5/24; A61P 13/00; A61P 23/00; A61P 25/04; A61P 17/00; A61P 15/00

(52) U.S. Cl. 514/170; 514/182; 514/171

(57) ABSTRACT

The present invention provides a pharmaceutical composition in the form of a water-based gel comprising: at least one pharmaceutically active compound; at least one gelling agent; a solubilising agent; and water, wherein said composition is free or substantially free of unsubstituted monohydric alcohols having between 1 and 6 carbon atoms. The invention also relates to methods for preparing the compositions and uses thereof.

### TECHNICAL FIELD

The present invention relates to water-based gel compositions comprising at least one pharmaceutically active compound, and also to methods of administering the compositions.

### BACKGROUND OF THE INVENTION

A major difficulty encountered with pharmaceutical compounds, particularly those of higher molecular weight, is that they are insoluble in aqueous solution. Because the bioavailability and hence efficacy of many pharmaceutical compounds is largely dependent on their presence in a soluble form, the formulation of water-insoluble compounds in aqueous delivery vehicles is problematic.

In the context of topical delivery vehicles this problem has been addressed by the incorporation of an organic solvent into the vehicle which improves the solubility of the water-insoluble compound(s), and hence increases bioavailability. Where the water-insoluble compounds are steroid hormones lower alcohols (i.e. alcohols having between 1 and 6 carbon atoms) are typically used to enhance solubility. However, lower alcohols such as ethanol have the undesired effect of drying skin as a result of solubilisation of the hydrophobic components thereof. In addition, stinging also occurs when lower alcohols come into contact with sensitive membranes such as the vagina. A further problem associated with the use of ethanol is that it cannot be included in halal medications.

There is therefore a need for compositions wherein water-insoluble compounds can be effectively solubilised in the absence of lower alcohols.

### SUMMARY OF THE INVENTION

In a first aspect, the present invention provides a pharmaceutical composition in the form of a water-based gel comprising:

(i) at least one pharmaceutically active compound;

(ii) at least one gelling agent;

(iii) a solubilising agent; and

(iv) water,

wherein said composition is free or substantially free of unsubstituted monohydric alcohols having between 1 and 6 carbon atoms.

The at least one pharmaceutically active compound may be present in the composition in an amount between about 0.0005% (w/w) and about 25% (w/w), or between about 0.001% (w/w) and about 5% (w/w).

The at least one pharmaceutically active compound may be a compound which is insoluble in water or sparingly soluble in water.

The composition may further comprise at least one pharmaceutically active compound which is soluble in water.

The at least one pharmaceutically active compound may be any compound which provides a therapeutic benefit or a cosmetic benefit to a subject.

The at least one pharmaceutically active compound may be a compound active in the gynaecological field, a compound useful in the treatment of epithelial tissue disorders (such as skin disorders), a compound useful in the control of infection, a compound useful in the treatment of inflammatory conditions, a compound useful in the treatment of sexual dysfunction, a compound useful in the treatment of urological disorders, a compound useful in anaesthesia, an analgesic compound, a compound which is an α-adrenergic receptor agonist, a hormone or a prohormone.

The hormone may be a sex hormone, a thyroid hormone or a growth hormone.

The sex hormone may be an estrogen (for example estriol), an androgen (for example testosterone) or a progestagen.

The hormone may be a hormone used in hormone replacement therapy.

In one embodiment, the hormone is a natural or synthetic estrogen, for example estriol, estrone or estradiol.

The composition may comprise at least two pharmaceutically active compounds selected from the group consisting of: a natural or synthetic estrogen, an analgesic compound, a compound useful in anaesthesia and an α-adrenergic receptor agonist.

The composition may comprise at least two pharmaceutically active compounds, wherein one of the pharmaceutically active compounds is a natural or synthetic estrogen, and the other pharmaceutically active compound(s) is/are selected from the group consisting of: an analgesic compound, a compound useful in anaesthesia and an α-adrenergic receptor agonist.

The analgesic compound may be tramadol, the compound useful in anaesthesia may be a—caine anaesthetic and the α-adrenergic receptor agonist may be clonidine.

The hormone or prohormone may be selected from the group consisting of: thyroxine, diiodothyrosine, melatonin, epinephrine, natural and synthetic estrogens, dehydroepiandrosterone, ketodehydroepiandrosterone, testosterone, progesterone and human growth hormone.

The at least one pharmaceutically active compound may be a steroidal compound.

The at least one gelling agent may be selected from the group consisting of: algae extracts, gums, polysaccharides, starches, pectins, hydrolysed proteins, cellulose derivatives and polymers comprising pendant carboxylic acid groups, or esters thereof, or comprising pendant anhydrides of dicarboxylic acid groups and block co-polymers based on ethylene oxide and/or propylene oxide. The gelling agent may be natural, synthetic or semi-synthetic.

In one embodiment, the gelling agent may be selected from the group consisting of: polymers comprising pendant carboxylic acid groups, or esters thereof, or comprising pendant anhydrides of dicarboxylic acid groups and block co-polymers based on ethylene oxide and/or propylene oxide.

The gelling agent may be a carbomer.

The carbomer may be a polymer of acrylic acid cross-linked with polyalkenyl ethers or divinyl glycol.

The carbomer may be a copolymer of acrylic acid and long chain alkyl acrylates crosslinked with polyalkenyl ethers.

The gelling agent may be present in the composition in an amount between about 0.01% (w/w) and about 50% (w/w), or between about 0.05% (w/w) and about 10% (w/w).

The solubilising agent may be selected from the group consisting of pyrrolidone or a derivative thereof, castor oil, polyethoxylated castor oil, diethylene glycol monoethyl ether, propylene glycol caprylate, propylene glycol mono caprylate, medium chain glycerides, 2-methacryloxyethylphosphonylcholine, cyclodextrins and derivatives thereof, lecithin, polysorbates, PEG-phospholipids, phospholipids, cholesterol-PEG and saturated polyglycolised C₈-C₁₀ glycerides.

The solubilising agent may be a non-alcoholic solubilising agent.

The solubilising agent may be an N-alkyl-pyrrolidone such as N-methylpyrrolidone.

The composition may comprise between about 30% (w/w) and about 90% (w/w) of water.

The composition may comprise between about 50% (w/w) and about 90% (w/w) of water.

The composition may be adapted for topical or parenteral administration. In one embodiment, the composition is a topical composition.

The composition may further comprise one or more emollients, moisturisers or humectants.

The composition may be adapted for gynaecological application, for example for application to the vaginal epithelial tissue.

The composition may be free or substantially free of monohydric alcohols having between 1 and 6 carbon atoms.

The composition may have a viscosity between about 40,000 and 70,000 mPa·s at 25° C.

In a second aspect, the present invention provides a method for preparing a pharmaceutical composition in the form of a water-based gel, the method comprising:

(i) admixing a pharmaceutically active compound and a solubilising agent;

(ii) adding water; and

(iii) adding a gelling agent and agitating the resulting mixture for a period of time sufficient to form a gel.

Step (i) may further comprise agitating the resulting mixture for a period of time sufficient to at least partially solubilise the pharmaceutically active compound.

Each of the components recited in the second aspect may be as defined in the first aspect.

In a third aspect, the present invention provides a method for topically administering at least one pharmaceutically active compound to a subject, the method comprising administering to an epithelial layer of a tissue or organ of the subject a composition of the first aspect.

The at least one pharmaceutically active compound may be a compound active in the gynecological field, for example an estrogen.

The subject may be a female.

The epithelial tissue may be vaginal epithelial tissue.

In a fourth aspect, the present invention provides a method for parenterally administering at least one pharmaceutically active compound to a subject, the method comprising injecting within tissue planes of a subject a composition of the first aspect.

### DEFINITIONS

In the context of the present specification, the terms “a” and “an” are used herein to refer to one or to more than one (i.e. to at least one) of the grammatical object of the article. By way of example, “an element” means one element or more than one element.

In the context of the present specification, the term “comprising” means “including principally but not necessarily solely”. Furthermore, variations of the word “comprising”, such as “comprise” and “comprises”, have correspondingly varied meanings.

In the context of the present specification, the term “unsubstituted monohydric alcohols having between 1 and 6 carbon atoms” is understood to mean compounds having a single hydroxy group and a total of between 1 and 6 carbon atoms, wherein the carbon atoms are not substituted with any other functional groups. The term excludes monohydric alcohol compounds having carbon chains interrupted by heteroatoms, for example oxygen and nitrogen. In one embodiment of the invention the unsubstituted monohydric alcohol having between 1 and 6 carbon atoms is ethanol.

In the context of the present specification, the term “ . . . or a derivative thereof” in relation to pyrrolidone includes pyrrolidone compounds having a C₁-C₁₀ alkyl or a C₁-C₆ alkyl group attached to the nitrogen and/or one or more C₁-C₁₀ alkyl groups or one or more C₁-C₆ alkyl groups attached to one or more of the carbon atoms of the pyrrolidone nucleus. Examples of pyrrolidone derivatives include, but are not limited to: N-methyl pyrrolidone, N-vinyl pyrrolidone, 1,4-dimethyl-2-pyrrolidone and 1-ethyl-5-propyl-2-pyrrolidone.

In the context of the present specification, the term “about” is understood to refer to a range of values that a person of skill in the art would consider equivalent to the recited value in the context of achieving the same function or result.

In the context of the present specification, the term “water-based” means that water is a, or the, major component of the composition.

In the context of the present specification, the terms “substantially soluble” and “substantially solubilise” mean that the majority of the pharmaceutically active compound is dissolved in the composition. For example, at least 60%, or at least 70%, or at least 80%, or at least 90%, or at least 95%, or at least 98%, or at least 99% of the pharmaceutically active compound may be dissolved in the composition.

In the context of the present specification, the term “gel” means a material comprising a continuous solid network that is assembled from particles or polymers embedded in an aqueous phase. The term “gel” also includes a composition that comprises at least one gelling agent described herein.

In the context of the present specification, the term “dissolved” means that all of the pharmaceutically active compound is solubilised in the composition.

In the context of the present specification, the term “therapeutic benefit” means that the compound or compounds to which it refers provide a beneficial effect in the treatment of a disease or condition, or any symptoms thereof, or in the prevention of a disease or condition in a subject, for example a human.

In the context of the present specification, the term “cosmetic benefit” means that the compound or compounds to which it refers provide a beneficial effect in relation to cleansing, beautifying, promoting attractiveness or altering the appearance of a subject, for example a human.

In the context of the present specification, the term “epithelial layer” means external and internal epithelial surfaces of the body.

In the context of the present specification, the term “non-alcoholic solubilising agent” means an agent which is free or substantially free of alcohols, including polyhydric alcohols.

In the context of the present specification, the term “substantially free” is understood to mean less than about 0.01%, or less than about 0.005%, or less than about 0.001% of the recited component.

In the context of the present specification, the term “carbomer” means homopolymers, copolymers and interpolymers based on an acrylic acid backbone which may or may not be cross-linked.

In the context of the present specification, the term “prohormone” is understood to mean a compound that can be converted into a hormone. For example, a compound that can be converted into a hormone within the body (i.e. in vivo).

### DETAILED DESCRIPTION OF THE INVENTION

The present invention is directed to a pharmaceutical composition in the form of a water-based gel comprising at least one pharmaceutically active compound, at least one gelling agent, a solubilising agent and water, wherein said composition is free or substantially free of unsubstituted monohydric alcohols having between 1 and 6 carbon atoms.

The present invention is based on the discovery by the inventors that pharmaceutically active compounds which are insoluble or sparingly soluble in water are able to be solubilised in a water-based gel composition in the presence of a gelling agent and a solubilising agent. Because the gel compositions of the present invention are free or substantially free of unsubstituted monohydric alcohols having between 1 and 6 carbon atoms, application to epithelial tissue does not result in a drying effect nor a stinging sensation when applied to sensitive membranes such as the vagina. Typically, the gel compositions of the invention are clear or transparent gels.

In one embodiment of the invention, the at least one pharmaceutically active compound may be substantially soluble or dissolved in the composition. The inclusion of the pharmaceutically active compound in a soluble or substantially soluble form in the composition may increase its bioavailability as compared to when the compound is present in a suspended form.

The at least one pharmaceutically active compound may be any compound which provides a therapeutic benefit or a cosmetic benefit to a subject, for example an animal such as a human.

The at least one pharmaceutically active compound may be a compound active in the gynaecological field, a compound useful in the treatment of epithelial tissue disorders (such as skin disorders), a compound useful in the control of infection, for example an anti-bacterial, anti-viral, anti-fungal or anti-protozoal compound, a compound useful in the treatment of inflammatory conditions, a compound useful in the treatment of sexual dysfunction, a compound useful in the treatment of urological disorders, a compound useful in anaesthesia, an analgesic compound, a compound which is an α-adrenergic receptor agonist, a hormone or a prohormone.

Examples of compounds active in the gynaecological field include, but are not limited to natural and synthetic estrogens such as estriol, estradiol, estrone, ethinyl estradiol, mestranol, dienestrol, quinestrol and diethylstilbestrol, progestagens such, as dienogest, gestodene, levonorgestrel, norethisterone, norgestimate, desogestrel, ethisterone, etonogestrel, gestonorone, lynestrenol, megestrol, medroxyprogesterone, norelgestromin and tibolone, selective estrogen receptor modulators such as tamoxifen, raloxifene, toremifene and clomiphene, compounds useful in the treatment of endometriosis such as danazol and triptorelin, compounds useful for inducing labour and/or cervical ripening such as oxytocin and misoprostol, spermicidal compounds and androgens such as testosterone. In one embodiment, the compounds active in the gynaecological field may be natural or synthetic estrogens. In another embodiment, the compounds useful in the gynaecological field may be selected from the group consisting of: estrone, estradiol, estriol, testosterone and progesterone.

Examples of compounds active in the treatment of sexual dysfunction or urological disorders include, but are not limited to clomipramine, phentolamine, apomorphine, papevarine and prostaglandin.

Examples of compounds useful in anaesthesia include local anaesthetics such as ester and amide anaesthetics, for example procaine, amethocaine (tetracaine), cocaine, lidocaine, prilocaine, bupivicaine, levobupivacaine, ropivacaine, mepivacaine, dibucaine and other—caine anaesthetics.

Examples of hormones include sex hormones, thyroid hormones and growth hormone, such as for example, thyroxine, diiodothyrosine, melatonin, epinephrine, natural and synthetic estrogens, dehydroepiandrosterone, ketodehydroepiandrosterone, testosterone, progesterone and human growth hormone.

Examples of analgesic compounds include narcotic and non-narcotic analgesics such as tramadol, acetominophen, ibuprofen, naproxen, buprenorphine, morphine; codeine, propoxyphene, fentanyl and amitriptyline.

Examples of α-adrenergic receptor agonists include: clonidine, guanfacine, methoxamine, oxymetazoline and guanabenz.

In one embodiment, the α-adrenergic receptor agonist is an α2-adrenergic receptor agonist.

Examples of anti-bacterial compounds include, but are not limited to antibiotics such as erythromycin, spiramycin, clarithromycin, clindamycin and tretinoin. Examples of anti-viral compounds include, but are not limited to acyclovir, amantadine, valacyclovir and rimantadine. Examples of anti-fungal compounds include, but are not limited to chlorphenesin, clioquinol, haloprogin, undecylenic acid, tolnaftate, fluconazole, butoconazole, clotrimazole, econazole, miconazole, terconazole and tioconazole. Examples of anti-protozoal compounds include, but are not limited to anti-malarial drugs, spiramycin and clioquinol.

Examples of compounds useful in the treatment of epithelial disorders include, but are not limited to steroidal compounds such as hydrocortisone.

Examples of compound useful in the treatment of inflammatory conditions include, but are not limited to NSADDS.

In another embodiment the at least one pharmaceutically active compound is a steroidal compound. Examples of steroidal compounds include, but are not limited to estradiol and esters thereof, ethinyl estradiol, conjugated estrogens, testosterone and esters thereof, cyproterone, drospirenone, etonogestrel, desogestrel, gestodene, levonorgestrel, norethisterones, norgestimate, norethindrone, norethindrone acetate, norethynodrel, norgestimate, norgestrel, medrogestone, medroxyprogesterone acetate, progesterone, spironolactones, eplerenone, canrenoate, canrenone, dicirenone, mexrenoate, prorenoate, epostane, mespirenone, oxprenoate, spirorenone, spiroxasone, prorenone, asoprisnil, beclomethasone dipropionate, betamethasone, betamethasone valerate, budesonide, clobetasol propionate, clobetasone butyrate, cortisone acetate, cortisol, dexamethasone, fludrocortisone acetate, prednisolone, prednisone, alfacalcidol, calcifediol, calciferol and calcitriol.

In an embodiment of the invention, the compositions comprise at least one pharmaceutically active compound that is insoluble in water or sparingly soluble in water, and at least one pharmaceutically active compound which is soluble in water.

The at least one pharmaceutically active compound may be present in the composition in an amount between about 0.0005% (w/w) and about 20% (w/w), or between about 0.0005% (w/w) and about 10% (w/w), or between about 0.005% (w/w) and about 5% (w/w), or between about 0.005% (w/w) and about 3% (w/w), or between about 0.005% (w/w) and about 1% (w/w), or between about 0.005% (w/w) and about 0.5% (w/w).

Gelling agents that may be used in the compositions of the invention include, but are not limited to: algae extracts, gums, polysaccharides, starches, pectins, hydrolysed proteins, cellulose derivatives, polymers comprising pendant carboxylic acid groups, or esters thereof, polymers comprising pendant anhydrides of dicarboxylic acid groups and block co-polymers based on ethylene oxide and/or propylene oxide.

Algae extracts that may be used include, but are not limited to alginates and carrageenans. Cellulose derivatives that may be used include, but are not limited to methylcelluloses, ethylcelluloses hydroxypropylmethylcelluloses, hydroxyethylcelluloses and carboxymethylcelluloses, which may or may not be cross-linked. Hydrolysed proteins include but are not limited to gelatin.

Polymers comprising pendant carboxylic acid groups may be homopolymers, copolymers or interpolymers comprising an acrylic acid backbone, for example carbomers. In one embodiment, the gelling agent is a polymer of acrylic acid cross-linked with polyalkenyl ethers or divinyl glycol. In an alternative embodiment, the gelling agent is a copolymer of acrylic acid and long-chain alkyl acrylates crosslinked with polyalkenyl ethers, for example allyl pentaerythritol.

Carbomers suitable for use in the present invention include, but are not limited to, those commercially available under the trade names Carbopol® (Lubrizol Advanced Materials, Inc.), Pemulen® (Lubrizol Advanced Materials, Inc.), Noveon® (Lubrizol Advanced Materials, Inc.), Synthalen® (3V Sigma) and Hivis Wako® (Wako Pure Chemicals Co.). Carbomers used in the present invention may be carbomers having Brookfield viscosities in the range of about 40,000 to 70,000 mPa·s at 25° C. In one embodiment, the carbomer is Carbopol®980.

Block co-polymers based on ethylene oxide and/or propylene oxide that are suitable for use in the present invention include those commercially available under the trade name Pluronic®. In one embodiment, the block co-polymer based on ethylene oxide and/or propylene oxide is Pluronic®F127 NF.

The amount of gelling agent present in the composition will depend on the particular gelling agent being used. Typically the amount of gelling agent present in the composition is between about 0.01% (w/w) and about 50% (w/w), or between about 0.05% (w/w) and about 40% (w/w), or between about 0.05% (w/w) and about 30% (w/w), or between about 0.05% (w/w) and about 20% (w/w), or between about 0.05% (w/w) and about 10% (w/w), or between about 0.05% (w/w) and about 5% (w/w), or between about 0.05% (w/w) and about 3% (w/w), or between about 0.1% (w/w) and about 2% (w/w). Where a gelling agent sold under the trade name Carbopol® is employed, the amount used may be in the range of between about 0.05% (w/w) and about 5% (w/w). Where a gelling agent sold under the trade name Pluronic® is employed, the amount used may be in the range of between about 1% (w/w) and about 40% (w/w).

The solubilising agent may be selected from the group consisting of: pyrrolidone or a derivative thereof, castor oil, polyethoxylated castor oil, diethylene glycol monoethyl ether, propylene glycol caprylate, propylene glycol mono caprylate, medium chain glycerides, 2-methacryloxyethylphosphonylcholine, cyclodextrins and derivatives thereof, lecithin, polysorbates, PEG-phospholipids, phospholipids, cholesterol-PEG, saturated polyglycolised C₈-C₁₀ glycerides. In one embodiment, the solubilising agent is a non-alcoholic solubilising agent, for example pyrrolidone or a derivative thereof.

The solubilising agent may be present in an amount sufficient to ensure that the pharmaceutically active compound is substantially soluble or dissolved in the composition. The amount of solubilising agent present in the composition will be dependent on the degree of insolubility of the pharmaceutically active compound(s). Those skilled in the art will, by routine trial and experimentation, be able to determine the amount of solubilising agent required to either dissolve or substantially solubilise the pharmaceutically active compound. The solubilising agent may be present in an amount between about 1% (w/w) and about 40% (w/w), or between about 1% (w/w) and about 30% (w/w), or between about 1% (w/w) and about 20% (w/w), or between about 1% (w/w) and about 15% (w/w), or between about 1% (w/w) and about 10% (w/w).

The compositions may comprise water in an amount between about 50% (w/w) and about 90% (w/w), or between about 60% (w/w) and about 80% (w/w).

The compositions may further comprise additional pharmaceutically acceptable excipients known in the art, for example diluents, adjuvants, humectants, emollients (moisturisers) and preservatives. The inclusion of humectants and emollients provide a moisturising effect to the topical compositions when applied repeatedly to the skin thereby further minimising any drying effect that the composition may impart when applied to sensitive membranes such as the vagina.

A wide variety of suitable emollients are known to those skilled in the art. See for example the International Cosmetic Ingredient Dictionary and Handbook, Eds. Wenninger and McEwen, The Cosmetic, Toiletry, and Fragrance Assoc., Washington, D.C., 7^{th} Edition, 1997. Emollients useful in the present invention include, but are not limited to: glycerin, propylene glycol (for example PEG300), sorbitol, lanolin, lanolin derivatives, polyethylene glycol, aloe vera, glucamate DOE 120, allantoin, alginates, monoester salts of sulfosuccinates, ceramides, and mixtures thereof.

Examples of humectants include, but are not limited to glycerol, sorbitol, polyethylene glycol, mono- and oligomeric sugars, natural extracts such as quillaia, lactic acid and urea.

Examples of preservatives include but are not limited to benzyl alcohol and parabens.

In an embodiment of the first aspect, the composition comprises:

(i) a pharmaceutically active compound which is an estrogen;

(ii) a gelling agent which is a carbomer;

(iii) a solubilising agent which is pyrrolidone or a derivative thereof; and

(iv) water.

The estrogen may be a natural or synthetic estrogen, and may be present in an amount between about 0.01% (w/w) and about 3% (w/w). The carbomer may be a polymer sold under the trade name Carbopol® and may be present in an amount between about 0.05% (w/w) and about 2% (w/w). The pyrrolidone or a derivative thereof may be N-methyl-2-pyrrolidone (for example the product sold under the trade name Pharmasolve® by International Specialty Products) and may be present in an amount between about 1% (w/w) and 20% (w/w). The composition may further comprise an emollient, for example aloe vera. The composition is free, or substantially free of unsubstituted monohydric alcohols having between 1 and 6 carbon atoms.

In an alternative embodiment of the first aspect, the composition comprises:

(i) an estrogen, clonidine and tramadol;

(ii) a gelling agent which is a block co-polymer based on ethylene oxide and/or propylene oxide;

(iii) a solubilising agent which is pyrrolidone or a derivative thereof; and

(iv) water

The estrogen may be a natural or synthetic estrogen. The estrogen, clonidine and tramadol may be present in amounts between about 0.005% (w/w) and about 10% (w/w). The block co-polymer based on ethylene oxide and/or propylene oxide may be a polymer sold under the trade name Pluronic® and may be present in an amount between about 1% (w/w) and about 30% (w/w). The pyrrolidone or a derivative thereof may be N-methyl-2-pyrrolidone (for example the product sold under the trade name Pharmasolve® by International Specialty Products) and may be present in an amount between about 1% (w/w) and 20% (w/w). The composition may further comprise an emollient, for example aloe vera. The composition is free, or substantially free of unsubstituted monohydric alcohols having between 1 and 6 carbon atoms.

In another embodiment of the first aspect, the composition comprises:

(i) an estrogen, tetracaine and clonidine;

(ii) a gelling agent which is a block co-polymer based on ethylene oxide and/or propylene oxide;

(iii) a solubilising agent which is pyrrolidone or a derivative thereof; and

(iv) water

The estrogen may be a natural or synthetic estrogen. The estrogen, tetracaine and clonidine may be present in amounts between about 0.005% (w/w) and about 10% (w/w). The block co-polymer based on ethylene oxide and/or propylene oxide may be a polymer sold under the trade name Pluronic® and may be present in an amount between about 1% (w/w) and about 30% (w/w). The pyrrolidone or a derivative thereof may be N-methyl-2-pyrrolidone (for example the product sold under the trade name Pharmasolve® by International Specialty Products) and may be present in an amount between about 1% (w/w) and about 20% (w/w). The composition may further comprise an emollient, for example aloe vera. The composition is free, or substantially free of unsubstituted monohydric alcohols having between 1 and 6 carbon atoms.

The present invention also relates, in a second aspect, to a method for preparing a pharmaceutical composition in the form of a water-based gel, the method comprising: (i) admixing a pharmaceutically active compound and a solubilising agent; (ii) adding water, and (iii) adding a gelling agent and agitating the resulting mixture for a period of time sufficient to form a gel. The method does not include addition of an unsubstituted monohydric alcohol having between 1 and 6 carbon atoms.

Step (i) may further comprise agitating the resulting mixture for a period of time sufficient to at least partially solubilise the pharmaceutically active compound. The agitating may be performed by standard methods known to those skilled in the art such as stirring, swirling and/or heating as required. In one embodiment agitation is performed until the pharmaceutically active compound is dissolved in the solubilising agent.

The method may further comprise admixing the solution obtained following step (i) or step (ii) with a mixture comprising at least one further pharmaceutically active compound which is soluble in water.

In an embodiment of the second aspect, the method comprises:

(i) admixing at least one pharmaceutically active compound which is insoluble in water or sparingly soluble in water and a solubilising agent;

(ii) admixing the mixture obtained in step (i) with a mixture comprising at least one further pharmaceutically active compound which is soluble in water;

(iii) admixing the mixture obtained in step (ii) with a gelling agent, and agitating the resulting mixture for a period of time sufficient to form a gel, wherein water is added as part of, or between, steps (i), and/or (ii), and/or (iii). The method does not include addition of an unsubstituted monohydric alcohol having between 1 and 6 carbon atoms.

The water may be added as part of, or between, steps (ii) and/or (iii).

The water may be added as part of step (ii) and/or (iii).

Each of the components recited above in connection with the second aspect may be as defined in the first aspect.

Where the composition is adapted for topical administration, the method may further comprise the addition of one or more emollients, moisturisers or humectants. The one or more emollients, moisturisers or humectants may be added during or after admixture of the pharmaceutically active compound and the solubilising agent, simultaneously when, before or after adding the water, and/or simultaneously when, before or after adding the gelling agent. In one embodiment, the one or more emollients, moisturisers or humectants are added before and after the gelling agent. The method may further comprise adding additional water after addition of the gelling agent. In one embodiment, multiple emollients, moisturisers or humectants are prepared separately and added prior to or after the addition of the gelling agent.

Where the compositions are adapted for parenteral administration, one or more non-toxic parenterally acceptable diluents or carriers may be added to the compositions, for example Ringer's solution, isotonic saline, glucose solution, distilled water or phosphate buffered saline.

The present invention is also directed to a method for topically administering at least one pharmaceutically active compound to a subject, the method comprising administering to an epithelial layer of a tissue or organ of the subject the composition of the first aspect. In one embodiment, the pharmaceutically active compound is a compound active in the gynaecological field, for example an estrogen, androgen or progestagen. The subject may be a human, and in one embodiment is a female. The epithelial tissue may be any epithelial tissue which is accessible on the body of the subject. In one embodiment, the epithelial tissue is the vaginal epithelial tissue. The gel compositions of the present invention are mucoadhesive and hence are effectively retained on mucosal surfaces for extended periods of time. Accordingly the gel compositions are effective at delivering pharmaceutically active compounds to mucosal epithelia.

The present invention is further directed to a method for parenterally administering a pharmaceutically active compound to a subject, the method comprising injecting within tissue planes of a subject a composition of the first aspect. The tissue planes may define a body cavity such as, but not limited to: joint cavities, synovial cavities, bursa, muscle compartments, the carpel tunnel or Alcock canal. The composition may be injected within a tissue plane so as to allow the pharmaceutically active compound to come into contact with synovia, tendon sheaths, fascia or neurovascular bundles.

In one embodiment, the method may involve injection of a local anaesthetic within the Alcock canal for blocking pudendal nerve pain. Injection of the gel compositions of the present invention will also result in a reduction in the stinging sensation associated with the injection because the compositions are free or substantially free of unsubstituted monohydric alcohols having between 1 and 6 carbon atoms. In addition, injection of a gel composition within tissue planes may, in addition to providing increased solubility and hence bio-availability, result in a longer retention time of the pharmaceutically active compound(s) at the desired site.

The invention will now be described in more detail, by way of illustration only, with respect to the following examples. The examples are intended to serve to illustrate this invention and should not be construed as limiting the generality of the disclosure of the description throughout this specification.

### EXAMPLES

### Example 1

### Water-Based Gel Compositions Comprising an Estrogen

Water-based gel compositions in accordance with the invention comprise the following components in the amounts specified:

### Example 1.1

| Component | Amount | |
|---|---|---|
| Estriol (E3) | 30 | mg |
| Pharmasolve ® | 8 | ml |
| Propylene Glycol | 5 | ml |
| Carbopol ® 980 | 0.6 | g |
| Aloe Vera Powder (freeze dried) | 0.2 | g |
| PEG 300 | 7.5 | ml |
| Glycerol | 7.5 | ml |
| Benzoyl alcohol | 100 | μl |

| | |
|---|---|
| Trolamine | 1 drop |
| Distilled Water | q.s to 100 ml |

### Example 1.2

| Component | Amount | |
|---|---|---|
| Estriol (E3) | 15 | mg |
| Pharmasolve ® | 4 | ml |
| Propylene Glycol | 3.5 | ml |
| Tetracaine USP | 1.12 | g |
| Aloe Vera Powder (freeze dried) | 0.2 | g |
| PEG 300 | 4.5 | ml |
| Glycerol | 4.0 | ml |
| Clonidine | 3.8 | mg |
| Benzoyl alcohol | 100 | μl |
| Pluronic ® (F127 NF) 30% solution | 80 | ml |
| 36% hydrochloric acid | 0.3 | ml |

| | |
|---|---|
| Distilled Water | q.s to 100 ml |

### Example 1.3

| Component | Amount | |
|---|---|---|
| Estriol (E3) | 15 | mg |
| Pharmasolve ® | 4 | ml |
| Propylene Glycol | 3.5 | ml |
| Tramadol HCl | 3.4 | g |
| Aloe Vera Powder (freeze dried) | 0.2 | g |
| PEG 300 | 4.5 | ml |
| Glycerol | 4.0 | ml |
| Clonidine | 3.8 | mg |
| Benzoyl alcohol | 100 | μl |
| Pluronic ® (F127 NF) 30% solution | 80 | ml |

| | |
|---|---|
| Distilled Water | q.s to 100 ml |

### Example 2

### Preparation of the Water-Based Gel Composition of Example 1.1

The water-based gel composition of Example 1.1 may be prepared by the following method:
1.1 Disperse 0.2 g Aloe Vera Powder in 20 ml of warm (50° C.) purified water. Continue stirring over heat until the powder has completely gelled.
1.2 Whilst mixing, add 7.5 ml of PEG 300 and 7.5 ml glycerol.
1.3 Weigh 30 mg estriol directly into a separate beaker.
1.4 Add 8 ml of Pharmasolve® and swirl until the estriol has completely dissolved.
1.5 Add 40 ml of purified water and agitate by mixing.
1.6 Whilst mixing add 5 ml of propylene glycol.
1.7 Continue mixing and sift the Carbopol® 980 powder into water making sure that no lumps are formed.
1.8 Stir until the Carbopol® 980 is completely dispersed in the water phase.
1.9 Add mixture prepared in step 1.2 and continue stirring until completely mixed.
1.10 Continue mixing and take volume to 100 ml with purified water.
1.11 Add one drop of Trolamine and continue to mix until Trolamine is evenly distributed in the gel. Increase mixing speed as gel increases in viscosity but avoid introducing too many air bubbles.
1.12 Pack into 100 ml opaque dispensing jar. Expiry date is estimated to be 6 months.

### Example 3

### Alternative Preparation of the Water-Based Gel Composition of Example 1.1

The water-based gel composition of Example 1.1 may also be prepared by the following method:

### 1. Preparation of Solution 1

1.1 Warm 100 ml of purified water to 50° C. Whilst stirring, disperse 1.0 g Aloe Vera Powder into the water. Continue stirring over heat until the powder has completely gelled.
1.2 Allow to cool then add 37.5 ml of PEG 300 and 37.5 ml glycerol. When completely dissolved add 250 μl of benzoyl alcohol.

### 2. Preparation of Solution 2

2.1 Accurately weigh 300 mg of pure estriol into a 250 ml beaker.
2.2 Add 80 ml of Pharmasolve® and stir until dissolved.
2.3 Transfer to a 100 ml amber bottle.

### 3. Preparation of Solution 3

3.1 Place 250 ml of purified water into a beaker.
3.2 Heat the water to 70° C. with continual stirring.
3.3 Gradually add 3 g of Carbopol® (via a sieve), and continue mixing until the Carbopol® is well dispersed in the water. Allow the solution to cool whilst continuing to mix.
3.4 Once cool, add 25 ml propylene glycol and 250 μl benzoyl alcohol to the mixture. Continue to mix until dispersed.

### 4. Preparation of the Gel Composition

4.1 Accurately transfer 35 ml of Solution 1 to a 200 ml glass beaker. Place the beaker under the Eka mixer and begin to mix.
4.2 Accurately transfer exactly 8.0 ml of Solution 2 to the beaker. Continue mixing to achieve a homogeneous mix.
4.3 Accurately transfer 55 ml of Solution 3 to the mix and continue stirring.
4.4 Add 1 drop of trolamine to the mix. NOTE: a gel will rapidly form so the beaker should be secured to prevent it rotating with the stirrer.
4.5 Transfer the prepared gel to an appropriate size dispensing jar. The shelf life of the gel is estimated to be 6 months.

### Example 4

### Preparation of the Water-Based Gel Composition of Example 1.2

The water-based gel composition of Example 1.2 may be prepared by the following method:

### 1. Preparation of Clonidine Stock Solution

1.1 Accurately weigh 110 mg Clonidene HCl into a 100 ml volumetric flask. Dilute to 100 ml with purified water. The solution contains 1.1 mg Clonidine HCl per ml, or 0.95 mg/ml Clonidine.

### 2. Preparation of Solution A

2.1 Accurately weigh 1.12 g of Tetracaine into a clean glass beaker.
2.2 Add 0.3 ml of 36% HCl followed by 4 ml of clonidiene stock solution prepared in 1 above.
2.3 Mix the solution. The tetracaine will begin to dissolve.
2.4 Add the propylene glycol, glycerol and PEG 300 and continue to mix until a clear solution is formed.
2.5 Add 4 ml of solution 2 (see Example 3 above) and 100 μl of benzoyl alcohol and mix well.
2.6 Weigh out 0.2 g of Aloe Vera powder, add to the solution prepared in 2.3 above and mix until the Aloe Vera powder is dissolved.
2.7 Place the solution in the refrigerator and allow to cool for 30 minutes.

### 3. Preparation of the Gel Composition

3.1 Remove the base from a 100 ml Topitec Jar and place both jar and base in refrigerator.
3.2 Measure 80 ml of cold 30% Pluronic® solution in a pre-cooled cylinder.
3.3 Add the Pluronic® solution to solution A (which has been cooled) prepared above stirring with a pre-cooled glass rod.
3.4 When well mixed, transfer the gel solution to the cold Topitec jar.
3.5 Place the jar and liquid mix on the bench and allow to warm to room temperature.
3.6 Once the solution has gelled (when the temperature exceeds about 15° C.), fit the base to the jar.
3.7 Expiry date is estimated to be 6 months. The gel should NOT be stored refrigerated.

### Example 5

### Preparation of the Water-Based Gel Composition of Example 1.3

The water-based gel composition of Example 1.3 may be prepared by the following method:

### 1. Preparation of Clonidine Stock Solution

1.1 Accurately weigh 110 mg of Clonidene HCl into a 100 ml volumetric flask. Dilute to 100 ml with purified water. The solution contains 1.1 mg Clonidine HCl per ml or 0.95 mg/ml Clonidine.

### 2. Preparation of Solution A

2.1 Transfer 4 ml of Clonidiene stock solution prepared in 1 above into a small clean glass beaker.
2.2 Add the propylene glycol, glycerol and PEG 300 and mix until a clear solution is formed.
2.3 Add 4 ml of solution 2 (see Example 3 above) and 100 μl of benzoyl alcohol, mix well.
2.4 Weight out 0.2 g of Aloe Vera powder, add to the solution prepared in 2.3 above and mix until the Aloe Vera powder is dissolved.
2.5 Weigh out 3.4 g Tramadol HCl (equivalent to 2.98 g of Tramadol free base) powder and add to the solution prepared in 2.4 above. Mix until all powder has dissolved and a clear solution is obtained.
2.6 Place the solution in the refrigerator and allow to cool for 30 minutes.

### 3. Preparation of the Gel Composition

3.1 Remove the base from a 100 ml Topitec Jar and place both jar and base in refrigerator.
3.2 Measure 80 ml of cold 30% Pluronic solution in a pre-cooled cylinder.
3.3 Add the Pluronic® solution to solution A (which has been cooled) prepared above stirring with a pre-cooled glass rod.
3.4 When well mixed, transfer the gel solution to the cold Topitec jar.
3.5 Place the jar and liquid mix on the bench and allow to warm to room temperature.
3.6 Once the solution has gelled (when the temperature exceeds about 15° C.), fit the is base to the jar.
3.7 Expiry date is estimated to be 6 months. The gel should NOT be stored refrigerated.

1. A pharmaceutical composition in the form of a clear water-based gel comprising:
(i) at least one pharmaceutically active compound_which is an estrogen, androgen or progestagen;
(ii) at least one gelling agent;
(iii) a solubilising agent which is N-methylpyrrolidone; and
(iv) water,
wherein said composition is free or substantially free of unsubstituted monohydric alcohols having between 1 and 6 carbon atoms.

2. The composition of claim 1, wherein the at least one pharmaceutically active compound is present in the composition in an amount between about 0.0005% (w/w) and about 25% (w/w).

3. The composition of claim 2, wherein the at least one pharmaceutically active compound is present in the composition in an amount between about 0.001% (w/w) and about 5% (w/w).

4. The composition of claim 1, further comprising at least one pharmaceutically active compound which is soluble in water.

5. The composition of claim 1, wherein the at least one pharmaceutically active compound is an estrogen.

6. The composition of claim 1, comprising at least two pharmaceutically active compounds selected from the group consisting of: a natural or synthetic estrogen, an analgesic compound, a compound useful in anaesthesia and an α-adrenergic receptor agonist.

7. The composition of claim 6, comprising at least two pharmaceutically active compounds, wherein one of the pharmaceutically active compounds is a natural or synthetic estrogen, and the other pharmaceutically active compound(s) is/are selected from the group consisting of: an analgesic compound, a compound useful in anaesthesia and an α-adrenergic receptor agonist.

8. The composition of claim 6, wherein the analgesic compound is amitriptyline.

9. The composition of claim 1, further comprising cortisone.

10. The composition of claim 6, wherein the analgesic compound is tramadol, the compound useful in anaesthesia is a—caine anaesthetic and the α-adrenergic receptor agonist is clonidine.

11. The composition of claim 1, wherein the gelling agent is selected from the group consisting of: algae extracts, gums, polysaccharides, starches, pectins, hydrolysed proteins, cellulose derivatives and polymers comprising pendant carboxylic acid groups, or esters thereof, polymers comprising pendant anhydrides of dicarboxylic acid groups and block co-polymers based on ethylene oxide and/or propylene oxide.

12. The composition of claim 11, wherein the gelling agent is selected from the group consisting of: polymers comprising pendant carboxylic acid groups, or esters thereof, or comprising pendant anhydrides of dicarboxylic acid groups and block co-polymers based on ethylene oxide and/or propylene oxide.

13. The composition of claim 12, wherein the polymer comprising pendant carboxylic acid groups is a carbomer.

14. The composition of claim 13, wherein the carbomer is a polymer of acrylic acid cross-linked with polyalkenyl ethers or divinyl glycol.

15. The composition of claim 13, wherein the carbomer is a copolymer of acrylic acid and long-chain alkyl acrylates crosslinked with polyalkenyl ethers.

16. The composition of claim 1, wherein the gelling agent is present in the composition in an amount between about 0.01% (w/w) and about 50% (w/w).

17. The composition of claim 1, wherein the composition has a viscosity between about 40,000 and 70,000 mPa·s at 25° C.

18. The composition of claim 1, which is a topical composition.

19. The composition of claim 18, wherein the composition comprises one or more emollients, moisturisers or humectants.

20. The composition of claim 1, wherein the composition is adapted for gynaecological application.

21. A method for preparing a pharmaceutical composition in the form of a clear,water-based gel, the method comprising:
(i) admixing an estrogen, androgen or progestagen and N-methylpyrrolidone;
(ii) adding water; and
(iii) adding a gelling agent and agitating the resulting mixture for a period of time sufficient to form a gel, wherein the method does not include addition of an unsubstituted monohydric alcohol having between 1 and 6 carbon atoms.

22. The method of claim 21, wherein step (i) further comprises agitating the mixture for a period of time sufficient to at least partially solubilise the estrogen, androgen or progestagen.

23. A method for topically administering at least one pharmaceutically active compound to a subject, the method comprising administering to an epithelial layer of a tissue or organ of the subject a composition of claim 1.

24. The method of claim 23, wherein the epithelial tissue is vaginal epithelial tissue.

25. A method for parenterally administering at least one pharmaceutically active compound to a subject, the method comprising injecting within tissue planes of a subject a composition of claim 1.
